(19) United States
(12) Patent Application Publication
(10) Pub. No.: US 2026/0315412 A1
(43) Pub. Date: Oct. 15, 2026

(54) TRIACIN-C PRODUCT

(71) Applicant: Anima Pharmaceuticals Pvt Ltd, Mumbai (IN)

(72) Inventor: Anup K. DAM, Ivyland, PA (US)

(73) Assignee: Anima Pharmaceuticals Pvt Ltd, Mumbai (IN)

(21) Appl. No.: 19/485,215

(22) PCT Filed: May 20, 2024
(86) PCT No.: PCT/US2024/030168

§ 371 Date: Nov. 17, 2025

Related U.S. Application Data
(60) Provisional application No. 63/467,407, filed on May 18, 2023.

(51) Int. Cl. A61K 31/137; A61K 9/00; A61K 31/4439; A61K 31/485; A61K 47/10; A61K 47/12; A61K 47/14; A61K 47/26

(52) U.S. Cl. CPC A61K 31/137; A61K 9/0095; A61K 31/4439; A61K 31/485; A61K 47/10; A61K 47/12; A61K 47/14; A61K 47/26

(57) ABSTRACT

Disclosed herein are oral pharmaceutical compositions comprising codeine phosphate, triprolidine hydrochloride, and pseudoephedrine hydrochloride, as well as methods of using the same.

### CROSS-REFERENCE TO RELATED APPLICATION

This application is a national phase application filed under 35 U.S.C. § 371 of International Patent Application No. PCT/US2024/030168, filed May 20, 2024, which claims priority to, and the benefit under 35 U.S.C. § 119(e) of U.S. Provisional Application Ser. No. 63/467,407, filed May 18, 2023, which are hereby incorporated by reference in their entirety.

### FIELD OF THE INVENTION

The present invention relates to oral pharmaceutical compositions comprising codeine phosphate, triprolidine hydrochloride, and pseudoephedrine hydrochloride, and methods of using the same.

### BACKGROUND

Triacin-c is a syrup for oral administration that contains codeine phosphate, triprolidine hydrochloride, and pseudoephedrine hydrochloride. This combination product is used to temporarily relieve cough and upper respiratory symptoms, including nasal congestion, associated with allergy or the common cold. Codeine probably exerts its antitussive activity by depressing the medullary (brain) cough center, thereby raising its threshold for incoming cough impulses. Triprolidine hydrochloride is an antihistamine and prevents histamine from eliciting typical immediate hypersensitivity responses in the nose, eyes, lungs and skin. Pseudoephedrine hydrochloride acts as an indirect sympathomimetic agent by stimulating sympathetic (adrenergic) nerve endings to release norepinephrine. Norepinephrine in turn stimulates alpha and beta receptors throughout the body. The action of pseudoephedrine hydrochloride is apparently more specific for the blood vessels of the upper respiratory tract and less specific for the blood vessels of the systemic circulation. The vasoconstriction elicited at these sites results in the shrinkage of swollen tissues in the sinuses and nasal passages.

There is a need for improved compositions containing codeine phosphate, triprolidine hydrochloride, and pseudoephedrine hydrochloride for relief of cough and upper respiratory symptoms.

### BRIEF SUMMARY

The disclosure provides, in a first aspect, a composition (e.g., syrup) comprising codeine phosphate, triprolidine hydrochloride, and pseudoephedrine hydrochloride. In some embodiments, codeine phosphate is present in an amount of 0.1% to 0.5%, e.g., 0.1% to 0.3%, by weight of the composition. In some embodiments, triprolidine hydrochloride is present in an amount of 0.01% to 0.05%, e.g., 0.01% to 0.03%, by weight of the composition. In some embodiments, pseudoephedrine hydrochloride is present in an amount of 0.3% to 0.9%, e.g., 0.5 to 0.7%, by weight of the composition.

In some embodiments, the composition comprises a humectant. In some embodiments, the humectant comprises glycerol and sorbitol. In some embodiments, the composition comprises 1 wt. %-5 wt. % glycerol and 4 wt. %-65 wt. % sorbitol. In some embodiments, the composition is free or substantially free of sucrose. In some embodiments, the composition is free or substantially free of sugar.

In some embodiments, the composition comprises the following ingredients:

codeine phosphate
triprolidine hydrochloride
pseudoephedrine hydrochloride
sorbitol
glycerol
saccharine, e.g., sodium saccharine
caramel flavor
sodium benzoate
methylparaben
ethanol
buffer (e.g., citrate buffer)
water

In some embodiments, the composition comprises the following ingredients by weight:

| | |
|---|---|
| codeine phosphate | 0.1-0.5% |
| triprolidine hydrochloride | 0.01-0.05% |
| pseudoephedrine hydrochloride | 0.3-0.9% |
| sorbitol | 4-65% |
| glycerol | 1-5% |
| saccharine, e.g., sodium saccharine | 0.01-0.4% |
| caramel flavor | 0.01-0.4% |
| sodium benzoate | 0.01-0.4% |
| methylparaben | 0.01-0.4% |
| ethanol | 1-6% |
| buffer (e.g., citrate buffer) | 0.1%-1% |
| water | 19.95%-93.45% |

In some embodiments, the composition comprises the following ingredients by weight:

| | |
|---|---|
| codeine phosphate | 0.1-0.5% |
| triprolidine hydrochloride | 0.01-0.05% |
| pseudoephedrine hydrochloride | 0.3-0.9% |
| sorbitol | 4-10% |
| glycerol | 1-5% |
| saccharine, e.g., sodium saccharine | 0.01-0.4% |
| caramel flavor | 0.01-0.4% |
| sodium benzoate | 0.01-0.4% |
| methylparaben | 0.01-0.4% |
| ethanol | 1-6% |
| buffer (e.g., citrate buffer) | 0.1%-1% |
| water | 74.95%-93.45% |

In some embodiments, the composition comprises the following ingredients by weight:

| | |
|---|---|
| codeine phosphate | 0.1-0.3% |
| triprolidine hydrochloride | 0.01-0.03% |
| pseudoephedrine hydrochloride | 0.5-0.7% |
| sorbitol | 6-8% |
| glycerol | 1-3% |
| saccharine, e.g., sodium saccharine | 0.05-0.2% |
| caramel flavor | 0.05-0.2% |
| sodium benzoate | 0.05-0.2% |
| methylparaben | 0.05-0.2% |
| ethanol | 2-4% |
| buffer (e.g., citrate buffer) | 0.2%-0.4% |
| water | 82.77%-89.99% |

In some embodiments, the composition comprises the following ingredients by weight:

| | |
|---|---|
| codeine phosphate | 0.1-0.5% |
| triprolidine hydrochloride | 0.01-0.05% |
| pseudoephedrine hydrochloride | 0.3-0.9% |
| sorbitol | 50-61% |
| glycerol | 1-5% |
| saccharine, e.g., sodium saccharine | 0.01-0.4% |
| caramel flavor | 0.01-0.4% |
| sodium benzoate | 0.01-0.4% |
| methylparaben | 0.01-0.4% |
| ethanol | 1-6% |
| buffer (e.g., citrate buffer) | 0.1%-1% |
| water | 23.95%-47.45% |

In some embodiments, the composition comprises the following ingredients by weight:

| | |
|---|---|
| codeine phosphate | 0.1-0.3% |
| triprolidine hydrochloride | 0.01-0.03% |
| pseudoephedrine hydrochloride | 0.5-0.7% |
| sorbitol | 55-61% |
| glycerol | 1-3% |
| saccharine, e.g., sodium saccharine | 0.05-0.2% |
| caramel flavor | 0.05-0.2% |
| sodium benzoate | 0.05-0.2% |
| methylparaben | 0.05-0.2% |
| ethanol | 2-4% |
| buffer (e.g., citrate buffer) | 0.2%-0.4% |
| water | 29.77%-40.99% |

In another aspect, the disclosure further provides a method of relieving cough and/or upper respiratory symptoms, including nasal congestion, associated with allergy or the common cold, comprising administering an effective amount of the composition as disclosed herein to a subject in need thereof orally. In some embodiments, the method relieves cough and/or upper respiratory symptoms temporarily. In some embodiments, the subject is a diabetic patient.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The present invention provides, in an aspect, a composition, e.g., an oral pharmaceutical composition (Composition 1.0) comprising codeine phosphate, triprolidine hydrochloride, and pseudoephedrine hydrochloride. For example, the invention includes:
1.1. Composition 1.0, wherein codeine phosphate is present in an amount of 0.1% to 0.5%, e.g., 0.1% to 0.3%, by weight of the composition.
1.2. Composition 1.0 or 1.1, wherein triprolidine hydrochloride is present in an amount of 0.01% to 0.05%, e.g., 0.01% to 0.03%, by weight of the composition.
1.3. Any of the preceding compositions, wherein pseudoephedrine hydrochloride is present in an amount of 0.3% to 0.9%, e.g., 0.5 to 0.7%, by weight of the composition.
1.4. Any of the preceding compositions, wherein the composition comprises a humectant, optionally wherein the humectant is selected from glycerol, sorbitol, sucrose, xylitol, propylene glycol and a combination thereof.
1.5. Composition 1.4, wherein the humectant comprises glycerol and sorbitol.
1.6. Composition 1.5, wherein the composition comprises 1 wt. %-5 wt. % glycerol and 4 wt. %-65 wt. % sorbitol.
1.7. Composition 1.6, wherein the composition comprises 1 wt. %-5 wt. % glycerol and 4 wt. %-10 wt. % sorbitol, optionally wherein the composition comprises 1 wt. %-3 wt. % glycerol and 6 wt. %-8 wt. % sorbitol.
1.8. Composition 1.6, wherein the composition comprises! wt. %-5 wt. % glycerol and 20 wt. %-65 wt. % sorbitol, optionally wherein the composition comprises 1 wt. %-5 wt. % glycerol and 20 wt. %-61 wt. %, e.g., 30 wt. %-61 wt. %, 40 wt. %-61 wt. %, 50 wt. %-61 wt. %, or 55 wt. %-61 wt. % sorbitol.
1.9. Composition 1.8, wherein the composition comprises 1 wt. %-3 wt. % glycerol and 20 wt. %-65 wt. % sorbitol, optionally wherein the composition comprises 1 wt. %-3 wt. % glycerol and 20 wt. %-61 wt. %, e.g., 30 wt. %-61 wt. %, 40 wt. %-61 wt. %, 50 wt. %-61 wt. %, or 55 wt. %-61 wt. % sorbitol.
1.10. Any of the preceding compositions, wherein the composition is free or substantially free of sucrose, optionally wherein the composition is free or substantially free of sugar.
1.11. Any of the preceding compositions, wherein the composition comprises a buffer, optionally wherein the buffer is selected from ascorbate buffer, citrate buffer, phosphate buffer, acetate buffer, carbonate buffer, and triethanolamine buffer.
1.12. Composition 1.11, wherein the buffer is citrate buffer, optionally wherein the citrate buffer comprises sodium citrate and citric acid.
1.13. Composition 1.11 or 1.12, wherein the buffer (e.g., citrate buffer) is present in an amount of 0.1% to 1% by weight of the composition, optionally wherein the buffer (e.g., citrate buffer) is present in an amount of 0.2% to 0.4% by weight of the composition.
1.14. Any of the preceding compositions, wherein the pH of the composition is between 4 to 5.5, optionally wherein the pH of the composition is between 4.1 to 5.1.
1.15. Any of the preceding compositions, wherein the composition comprises a preservative, optionally wherein the preservative is selected from benzalkonium chloride, benzethonium chloride, benzoic acid, benzyl alcohol, butylparaben, cetylpyridinium chloride, chlorobutanol, chlorocresol, cresol, dehydroacetic acid, diazolidinyl urea, ethylparaben, methylparaben, phenylethyl alcohol, phenylmercuric acetate, phenylmercuric nitrate, potassium benzoate, potassium sorbate, propylparaben, sodium benzoate, sodium dehydroacetate, sodium propionate, sorbic acid, thimerosal, thymol, and a combination thereof.
1.16. Composition 1.15, wherein the composition comprises methylparaben and sodium benzoate.
1.17. Composition 1.16, wherein the composition comprises 0.01 wt. %-0.4 wt. % methylparaben and 0.01 wt. %-0.4 wt. % sodium benzoate, optionally wherein the composition comprises 0.05 wt. %-0.2 wt. % methylparaben and 0.05 wt. %-0.2 wt. % sodium benzoate.
1.18. Any of the preceding compositions, wherein the composition comprises a sweetener, optionally wherein the sweetener is selected from saccharin (e.g., sodium saccharin), acesulfame, neotame, cyclamate, sucralose, thaumatin, stevioside, glycyrrhizin, xylitol, maltitol, mannitol, and a combination thereof.
1.19. Composition 1.18, wherein the composition comprises saccharin, e.g., sodium saccharin.
1.20. Composition 1.19, wherein saccharine, e.g., sodium saccharine, is present in an amount of 0.01% to 0.4% by weight of the composition, optionally wherein saccharine, e.g., sodium saccharine, is present in an amount of 0.05% to 0.2% by weight of the composition.
1.21. Any of the preceding compositions, wherein the composition comprises a flavor, optionally wherein the flavor is selected from a mixed berry flavor, a mulberry flavor, a cherry flavor, a strawberry flavor, a citrus flavor, a lemon flavor, a lime flavor, an orange flavor, a grape flavor, a vanilla flavor, a chocolate flavor, a caramel flavor, a mint flavor, a spearmint flavor, a wintergreen flavor, a menthol flavor, and a combination thereof.
1.22. Composition 1.21, wherein the composition comprises a caramel flavor, optionally wherein the caramel flavor is a sugar-free caramel flavor.
1.23. Composition 1.21 or 1.22, wherein the flavor, e.g., caramel flavor (e.g., sugar-free caramel flavor) is present in an amount of 0.01% to 0.4% by weight of the composition, optionally wherein the flavor, e.g., caramel flavor (e.g., sugar-free caramel flavor) is present in an amount of 0.05% to 0.2% by weight of the composition.
1.24. Any of the preceding compositions, wherein the composition comprises ethanol.
1.25. Composition 1.24, wherein ethanol is present in an amount of 1% to 6% by weight of the composition, optionally wherein ethanol is present in an amount of 2% to 4% by weight of the composition.
1.26. Any of the preceding compositions, wherein the composition comprises water.
1.27. Composition 1.26, wherein water is present in an amount of 70% to 95% by weight of the composition.
1.28. Any of the preceding compositions, wherein the composition is an oral pharmaceutical composition.
1.29. Any of the preceding compositions, wherein the composition is a syrup.
1.30. Any of the preceding compositions, wherein the composition comprises the following ingredients:

codeine phosphate
triprolidine hydrochloride
pseudoephedrine hydrochloride
sorbitol
glycerol
saccharine, e.g., sodium saccharine
caramel flavor
sodium benzoate
methylparaben
ethanol
buffer (e.g., citrate buffer)
water

1.31. Any of the preceding compositions, wherein the composition comprises the following ingredients by weight:

| | |
|---|---|
| codeine phosphate | 0.1-0.5% |
| triprolidine hydrochloride | 0.01-0.05% |
| pseudoephedrine hydrochloride | 0.3-0.9% |
| sorbitol | 4-65% |
| glycerol | 1-5% |
| saccharine, e.g., sodium saccharine | 0.01-0.4% |
| caramel flavor | 0.01-0.4% |
| sodium benzoate | 0.01-0.4% |
| methylparaben | 0.01-0.4% |
| ethanol | 1-6% |
| buffer (e.g., citrate buffer) | 0.1%-1% |
| water | 19.95%-93.45% |

1.32. Any of the preceding compositions, wherein the composition comprises the following ingredients by weight:

| | |
|---|---|
| codeine phosphate | 0.1-0.5% |
| triprolidine hydrochloride | 0.01-0.05% |
| pseudoephedrine hydrochloride | 0.3-0.9% |
| sorbitol | 4-10% |
| glycerol | 1-5% |
| saccharine, e.g., sodium saccharine | 0.01-0.4% |
| caramel flavor | 0.01-0.4% |
| sodium benzoate | 0.01-0.4% |
| methylparaben | 0.01-0.4% |
| ethanol | 1-6% |
| buffer (e.g., citrate buffer) | 0.1%-1% |
| water | 74.95%-93.45% |

1.33. Any of the preceding compositions, wherein the composition comprises the following ingredients by weight:

| | |
|---|---|
| codeine phosphate | 0.1-0.3% |
| triprolidine hydrochloride | 0.01-0.03% |
| pseudoephedrine hydrochloride | 0.5-0.7% |
| sorbitol | 6-8% |
| glycerol | 1-3% |
| saccharine, e.g., sodium saccharine | 0.05-0.2% |
| caramel flavor | 0.05-0.2% |
| sodium benzoate | 0.05-0.2% |
| methylparaben | 0.05-0.2% |
| ethanol | 2-4% |
| buffer (e.g., citrate buffer) | 0.2%-0.4% |
| water | 82.77%-89.99% |

1.34. Any of the preceding compositions, wherein the composition comprises the following ingredients by weight:

| | |
|---|---|
| codeine phosphate | 0.1-0.5% |
| triprolidine hydrochloride | 0.01-0.05% |
| pseudoephedrine hydrochloride | 0.3-0.9% |
| sorbitol | 50-61% |
| glycerol | 1-5% |
| saccharine, e.g., sodium saccharine | 0.01-0.4% |
| caramel flavor | 0.01-0.4% |
| sodium benzoate | 0.01-0.4% |
| methylparaben | 0.01-0.4% |
| ethanol | 1-6% |
| buffer (e.g., citrate buffer) | 0.1%-1% |
| water | 23.95%-47.45% |

1.35. Any of the preceding compositions, wherein the composition comprises the following ingredients by weight:

| | |
|---|---|
| codeine phosphate | 0.1-0.3% |
| triprolidine hydrochloride | 0.01-0.03% |
| pseudoephedrine hydrochloride | 0.5-0.7% |
| sorbitol | 55-61% |
| glycerol | 1-3% |
| saccharine, e.g., sodium saccharine | 0.05-0.2% |
| caramel flavor | 0.05-0.2% |
| sodium benzoate | 0.05-0.2% |
| methylparaben | 0.05-0.2% |
| ethanol | 2-4% |
| buffer (e.g., citrate buffer) | 0.2%-0.4% |
| water | 29.77%-40.99% |

1.36. Any of the preceding compositions for use to relieve cough and/or upper respiratory symptoms, including nasal congestion, associated with allergy or the common cold.

The composition of the present invention may be an oral pharmaceutical composition. In some embodiments, the composition is a syrup. For example, the active ingredients (codeine phosphate, triprolidine hydrochloride, and pseudoephedrine hydrochloride) may be supplied in a colorless, caramel flavored vehicle in the form of syrup in 16 fl oz (473 mL) size bottle. In certain embodiments, each 5 mb (one teaspoonful) of syrup for oral administration contains 10 mg codeine phosphate, 1.25 mg triprolidine hydrochloride and 30 mg pseudoephedrine hydrochloride. Dosage may be individualized according to the needs and response of the patient. For example, Dosage may be determined by age. The usual dose for adults and children 12 years and older may be 2 teaspoonfuls (10 m) every 4 to 6 hours, not to exceed 8 teaspoonfuls (40 mL) in 24 hours. The usual dose for children 6 to under 12 years may be 1 teaspoonful (5 mL) every 4 to 6 hours, not to exceed 4 teaspoonfuls (20 mL) in 24 hours. The usual dose for Children 2 to under 6 years may be V2 teaspoonful (2.5 mL) every 4 to 6 hours, not to exceed 2 teaspoonfuls (10 mL).

The composition of the present invention may contain a pharmaceutically acceptable carrier. As used herein, a “pharmaceutically acceptable carrier” refers to a material or combination of materials that are safe for use in the compositions of the invention, commensurate with a reasonable benefit/risk ratio. Such materials include but are not limited to, for example, water, humectants, buffering agents. Such materials are well known in the art and are readily chosen by one skilled in the art based on the physical and aesthetic properties desired for the compositions being prepared. In some embodiment, the pharmaceutically acceptable carrier may include a pharmaceutically acceptable solvent. Illustrative solvents may include, but are not limited to, one or more of alcohol, e.g., ethanol, and water. In a particular embodiment, the pharmaceutically acceptable solvent includes water and ethanol. Water employed in the preparation of commercial oral compositions should be deionized and free of organic impurities. Water commonly makes up the balance of the compositions and includes about 70% to about 95%, by weight of the compositions. This amount of water includes the free water which is added plus that amount which is introduced with other materials such as with sorbitol or any components of the invention. Alcohol, e.g., ethanol may be present in an amount of 1% to 6%, e.g., 2% to 4%, by weight of the composition.

The composition of the present invention contains codeine phosphate. Codeine is an opiate and an agonist of the mu opioid receptor (MOR). Codeine is readily absorbed from the gastrointestinal tract, with a therapeutic dose reaching peak antitussive effectiveness in about 2 hours and persisting for 4 to 6 hours. Codeine is rapidly distributed from blood to body tissues and taken up preferentially by parenchymatous organs such as liver, spleen and kidney. It passes the blood brain barrier and is found in fetal tissue and breast milk. Codeine is metabolized in the liver to morphine and norcodeine, each representing about 10 percent of the administered codeine dose. Morphine is ten times more potent against the mu receptor than codeine. Opioid receptors are G protein-coupled receptors that positively and negatively regulate synaptic transmission through downstream signaling. Binding of codeine or morphine to the mu opioid receptor results in hyperpolarization of the neuron leading to the inhibition of release of nociceptive neurotransmitters, causing an analgesic effect and increased pain tolerance due to reduced neuronal excitability. Codeine probably exerts its antitussive activity by depressing the medullary (brain) cough center, thereby raising its threshold for incoming cough impulses. In some embodiments, codeine phosphate is present in an amount of 0.1% to 0.5% by weight of the composition. In some embodiments, codeine phosphate is present in an amount of 0.1% to 0.3% by weight of the composition.

The composition of the present invention contains triprolidine hydrochloride. Triprolidine hydrochloride is an antihistamine and prevents histamine from eliciting typical immediate hypersensitivity responses in the nose, eyes, lungs and skin. Therefore, triprolidine is used to relieve symptoms of allergy, hay fever, and the common cold. In some embodiments, triprolidine hydrochloride is present in an amount of 0.01% to 0.05% by weight of the composition. In some embodiments, triprolidine hydrochloride is present in an amount of 0.01% to 0.03% by weight of the composition.

The composition of the present invention contains pseudoephedrine hydrochloride. Pseudoephedrine is a sympathomimetic drug of the phenethylamine and amphetamine chemical classes. It is used as a nasal/sinus decongestant, a stimulant, or a wakefulneess-promoting agent. Pseudoephedrine is rapidly and almost completely absorbed from the gastrointestinal tract. Pseudoephedrine is distributed to body tissues and fluids, including fetal tissue, breast milk and the central nervous system (CNS). Pseudoephedrine acts as an indirect sympathomimetic agent by stimulating sympathetic (adrenergic) nerve endings to release norepinephrine. Norepinephrine in turn stimulates alpha and beta receptors throughout the body. The action of pseudoephedrine hydrochloride is apparently more specific for the blood vessels of the upper respiratory tract and less specific for the blood vessels of the systemic circulation. The vasoconstriction elicited at these sites results in the shrinkage of swollen tissues in the sinuses and nasal passages. In some embodiments, pseudoephedrine hydrochloride is present in an amount of 0.3% to 0.9% by weight of the composition. In some embodiments, pseudoephedrine hydrochloride is present in an amount of 0.5 to 0.7% by weight of the composition.

The composition of the present invention may include one or more humectants. Humectants can reduce evaporation and also contribute towards preservation by lowering water activity and can also impart desirable sweetness or flavor to compositions. Suitable humectants include edible polyhydric alcohols such as glycerol, sorbitol, sucrose, xylitol, propylene glycol as well as other polyols and mixtures of these humectants. The use of sugar, e.g., sucrose as a humectant may lead to significant degradation of triprolidine. In the present invention, it has been found that this problem can be solved by using sorbitol in place of sucrose. Thus, in some embodiments, the composition comprises glycerol and sorbitol. In some embodiments, the composition comprises 1 wt. %-5 wt. % glycerol and 4 wt. %-65 wt. % sorbitol. In some embodiments, the composition comprises 1 wt. %-5 wt. % glycerol and 4 wt. %-10 wt. % sorbitol. In some embodiments, the composition comprises 1 wt. %-3 wt. % glycerol and 6 wt. %-8 wt. % sorbitol. In some embodiments, the composition comprises! wt. %-5 wt. % glycerol and 20 wt. %-65 wt. % sorbitol. In some embodiments, the composition comprises 1 wt. %-5 wt. % glycerol and 20 wt. %-61 wt. %, e.g., 30 wt. %-61 wt. %, 40 wt. %-61 wt. %, 50 wt. %-61 wt. %, or 55 wt. %-61 wt. % sorbitol. In some embodiments, the composition is free or substantially free of sucrose. In some embodiments, the composition is free or substantially free of sugar.

The composition of the present invention may include a buffer configured to control or modulate the pH within a predetermined or desired range. Illustrative buffer may include, but are not limited to, ascorbate buffer, citrate buffer, phosphate buffer, acetate buffer, carbonate buffer, and triethanolamine buffer. In some embodiments, the buffer is citrate buffer. In some embodiment, the citrate buffer comprises sodium citrate and citric acid. In some embodiments, the buffer, e.g., citrate buffer is present in an amount of 0.1% to 1% by weight of the composition. In some embodiment, the buffer, e.g., citrate buffer is present in an amount of 0.2% to 0.4% by weight of the composition. In some embodiments, the pH of the composition is between 4 and 5.5. In some embodiments, the pH of the composition is between 4.1 and 5.1.

The composition of the present invention may include one or mor preservatives. Suitable preservatives include, but are not limited to, benzalkonium chloride, benzethonium chloride, benzoic acid, benzyl alcohol, butylparaben, cetylpyridinium chloride, chlorobutanol, chlorocresol, cresol, dehydroacetic acid, diazolidinyl urea, ethylparaben, methylparaben, phenylethyl alcohol, phenylmercuric acetate, phenylmercuric nitrate, potassium benzoate, potassium sorbate, propylparaben, sodium benzoate, sodium dehydroacetate, sodium propionate, sorbic acid, thimerosal, and thymol. In some embodiments, the composition comprises methylparaben and sodium benzoate. In some embodiments, the composition comprises 0.01 wt. %-0.4 wt. % methylparaben and 0.01 wt. %-0.4 wt. % sodium benzoate. In some embodiments, the composition comprises 0.05 wt. %-0.2 wt. % methylparaben and 0.05 wt. %-0.2 wt. % sodium benzoate.

The composition of the present invention may include a sweetener such as saccharin, for example sodium saccharin, acesulfame, neotame, cyclamate or sucralose, thaumatin, stevioside, glycyrrhizin, xylitol, maltitol or mannitol. In some embodiments, the sweetener is saccharin, e.g., sodium saccharin. In some embodiments, saccharine, e.g., sodium saccharine is present in an amount of 0.01% to 0.4% by weight of the composition. In some embodiments, saccharine, e.g., sodium saccharine is present in an amount of 0.05% to 0.2% by weight of the composition.

The composition of the present invention may include a flavor. Suitable flavors include, but are not limited to, a mixed berry flavor, a mulberry flavor, a cherry flavor, a strawberry flavor, a citrus flavor, a lemon flavor, a lime flavor, an orange flavor, a grape flavor, a vanilla flavor, a chocolate flavor, a caramel flavor, a mint flavor, a spearmint flavor, a wintergreen flavor, a menthol flavor, and any combinations thereof. In some embodiments, the composition comprises a caramel flavor. In some embodiments, the caramel flavor is a sugar-free caramel flavor. In some embodiments, the flavor, e.g., caramel flavor (e.g., sugar-free caramel flavor) is present in an amount of 0.01% to 0.4% by weight of the composition. In some embodiments, the flavor, e.g., caramel flavor (e.g., sugar-free caramel flavor) is present in an amount of 0.05% to 0.2% by weight of the composition.

In another aspect, the invention further provides a method of relieving cough and/or upper respiratory symptoms, including nasal congestion, associated with allergy or the common cold, comprising administering an effective amount of the composition as disclosed herein to a subject in need thereof orally. As used herein, the term “effective amount” is intended to encompass a therapeutically effective amount to relieve cough and/or upper respiratory symptoms. In some embodiments, the method relieves cough and/or upper respiratory symptoms, including nasal congestion, associated with allergy or the common cold temporarily. In some embodiments, the composition is a syrup. Syrup formulations containing sucrose is not a good option for diabetic patients. In the present invention, sucrose is replaced with sorbitol. Sorbitol is not concerned for diabetic patients. In some embodiments, the subject is a diabetic patient.

### EXAMPLES

### Example 1

A stability study is performed in which the stability of triprolidine HCl is compared between three solutions. Three solutions containing triprolidine hydrochloride are prepared as shown in Table 1.

TABLE 1

| | | stability of triprolidine | |
|---|---|---|---|
| | | HCl (% of LC) | |
| | | | 3 days at |
| | Ingredients | Initial | 70° C. |
| Solution 1 | triprolidin + water | 101.60% | 98.50% |
| Solution 2 | triprolidin + water + | 102.10% | 84.80% |
| | sucrose syrup | | |
| Solution 3 | triprolidin + water + | 98.80% | 98.30% |
| | non-crystalline sorbitol (70%) | | |

Solution 1 is a water solution containing triprolidine HCl. Solution 2 contains triprolidine HCl and sucrose syrup, while solution 3 contains triprolidine HCl and sorbitol. The stability of triprolidine HCl in the three solutions is examined by measuring % of LC after accelerated aging at 70° C. for 3 days. As shown in Table 1, the stability of triprolidine HCl is improved with use of sorbitol (solution 3), compared to sucrose (solution 2). This result shows that sucrose accelerates the degradation of triprolidine in liquid formulation and the replacement of sucrose with sorbitol improves the stability of triprolidine in liquid formulation.

### Example 2

Syrup formulations containing codeine phosphate, triprolidine hydrochloride, and pseudoephedrine hydrochloride are prepared with the following ingredients:

TABLE 2

| | Formulation 1 | Formulation 2 |
|---|---|---|
| ingredients | (weight %) | (weight %) |
| codeine phosphate | 0.20% | 0.20% |
| triprolidine hydrochloride | 0.03% | 0.03% |
| pseudoephedrine hydrochloride | 0.5% | 0.5% |
| sorbitol solution (70%) | 10% | 86.8% |
| glycerol | 2.53% | 2.53% |
| sodium saccharin | 0.08% | 0.08% |
| caramel flavor | 0.10% | 0.10% |
| sodium benzoate | 0.10% | 0.10% |
| methylparaben | 0.15% | 0.15% |
| Ethanol (95%) | 3.69% | 3.69% |
| citric acid | 0.12% | 0.12% |
| sodium citrate | 0.16% | 0.16% |
| water | q.s. | q.s. |

1. A composition comprising codeine phosphate, triprolidine hydrochloride, and pseudoephedrine hydrochloride.

2. The composition of claim 1, wherein codeine phosphate is present in an amount of 0.1% to 0.5% by weight of the composition.

3. The composition of claim 1, wherein triprolidine hydrochloride is present in an amount of 0.01% to 0.05% by weight of the composition.

4. The composition of claim 1, wherein pseudoephedrine hydrochloride is present in an amount of 0.3% to 0.9% by weight of the composition.

5. The composition of claim 1, wherein the composition comprises a humectant, optionally wherein the humectant is selected from glycerol, sorbitol, xylitol, propylene glycol and a combination thereof.

6. The composition of claim 5, wherein the composition comprises glycerol and sorbitol, optionally wherein the composition comprises 1 wt. %-5 wt. % glycerol and 4 wt. %-65 wt. % sorbitol.

7. The composition of claim 1, wherein the composition comprises a buffer, optionally wherein the buffer is selected from the group consisting of ascorbate buffer, citrate buffer, phosphate buffer, acetate buffer, carbonate buffer, and triethanolamine buffer.

8. The composition of claim 7, wherein the buffer is citrate buffer, optionally wherein the buffer is present in an amount of 0.1% to 1% by weight of the composition.

9. The composition of claim 1, wherein the composition comprises a preservative, optionally wherein the preservative is selected from the group consisting of benzalkonium chloride, benzethonium chloride, benzoic acid, benzyl alcohol, butylparaben, cetylpyridinium chloride, chlorobutanol, chlorocresol, cresol, dehydroacetic acid, diazolidinyl urea, ethylparaben, methylparaben, phenylethyl alcohol, phenylmercuric acetate, phenylmercuric nitrate, potassium benzoate, potassium sorbate, propylparaben, sodium benzoate, sodium dehydroacetate, sodium propionate, sorbic acid, thimerosal, thymol, and combinations thereof.

10. The composition of claim 9, wherein the composition comprises methylparaben and sodium benzoate, optionally wherein the composition comprises 0.01 wt. %-0.4 wt. % methylparaben and 0.01 wt. %-0.4 wt. % sodium benzoate.

11. The composition of claim 1, wherein the composition comprises a sweetener, optionally wherein the sweetener is selected from the group consisting of saccharin (e.g., sodium saccharin), acesulfame, neotame, cyclamate, sucralose, thaumatin, stevioside, glycyrrhizin, xylitol, maltitol, mannitol, and combinations thereof.

12. The composition of claim 11, wherein the sweetener is a saccharin, optionally wherein the saccharine is present in an amount of 0.01% to 0.4% by weight of the composition.

13. The composition of claim 1, wherein the composition comprises a flavor, optionally wherein the flavor is present in an amount of 0.01% to 0.4% by weight of the composition.

14. The composition of claim 13, wherein the flavor is a caramel flavor, optionally wherein the caramel flavor is a sugar-free caramel flavor.

15. The composition according to claim 1, wherein the composition comprises ethanol, optionally wherein ethanol is present in an amount of 1% to 6% by weight of the composition.

16. The composition of claim 1, wherein the composition is a syrup.

17. The composition of claim 1, wherein the composition is free or substantially free of sucrose, optionally wherein the composition is free or substantially free of sugar.

18. The composition of claim 1, wherein the composition comprises the following ingredients:
codeine phosphate
triprolidine hydrochloride
pseudoephedrine hydrochloride
sorbitol
glycerol
saccharine, [symbol] [symbol]
caramel flavor
sodium benzoate
methylparaben
ethanol
buffer [symbol] [symbol]
water

19. The composition of claim 1, wherein the composition comprises the following ingredients by weight:
A.

| | | |
|---|---|---|
| codeine phosphate | 0.1-0.5% | |
| triprolidine hydrochloride | 0.01-0.05% | |
| pseudoephedrine hydrochloride | 0.3-0.9% | |
| sorbitol | 4-65% | |
| glycerol | 1-5% | |
| saccharine, [symbol] [symbol] | 0.01-0.4% | |
| caramel flavor | 0.01-0.4% | |
| sodium benzoate | 0.01-0.4% | |
| methylparaben | 0.01-0.4% | |
| ethanol | 1-6% | |
| buffer [symbol] [symbol] | 0.1[[%]]-1% | |
| water | 19.95[[%]]-93.45% | , or |

B.

| | | |
|---|---|---|
| codeine phosphate | 0.1-0.5% | |
| triprolidine hydrochloride | 0.01-0.05% | |
| pseudoephedrine hydrochloride | 0.3-0.9% | |
| sorbitol | 4-10% | |
| glycerol | 1-5% | |
| saccharine | 0.01-0.4% | |
| caramel flavor | 0.01-0.4% | |
| sodium benzoate | 0.01-0.4% | |
| methylparaben | 0.01-0.4% | |
| ethanol | 1-6% | |
| buffer | 0.1-1% | |
| water | 74.95-93.45% | , or |

C.

| | | |
|---|---|---|
| codeine phosphate | 0.1-0.3% | |
| triprolidine hydrochloride | 0.01-0.03% | |
| pseudoephedrine hydrochloride | 0.5-0.7% | |
| sorbitol | 6-8% | |
| glycerol | 1-3% | |
| saccharine | 0.05-0.2% | |
| caramel flavor | 0.05-0.2% | |
| sodium benzoate | 0.05-0.2% | |
| methylparaben | 0.05-0.2% | |
| ethanol | 2-4% | |
| buffer | 0.2-0.4% | |
| water | 82.77-89.99% | , or |

D.

| | | |
|---|---|---|
| codeine phosphate | 0.1-0.5% | |
| triprolidine hydrochloride | 0.01-0.05% | |
| pseudoephedrine hydrochloride | 0.3-0.9% | |
| sorbitol | 50-61% | |
| glycerol | 1-5% | |
| saccharine | 0.01-0.4% | |
| caramel flavor | 0.01-0.4% | |
| sodium benzoate | 0.01-0.4% | |
| methylparaben | 0.01-0.4% | |
| ethanol | 1-6% | |
| buffer | 0.1-1% | |
| water | 23.95-47.45% | , or |

E.

| | | |
|---|---|---|
| codeine phosphate | 0.1-0.3% | |
| triprolidine hydrochloride | 0.01-0.03% | |
| pseudoephedrine hydrochloride | 0.5-0.7% | |
| sorbitol | 55-61% | |
| glycerol | 1-3% | |
| saccharine | 0.05-0.2% | |
| caramel flavor | 0.05-0.2% | |
| sodium benzoate | 0.05-0.2% | |
| methylparaben | 0.05-0.2% | |
| ethanol | 2-4% | |
| buffer | 0.2-0.4% | |
| water | 29.77-40.99% | . |

20-23. (canceled)

24. A method of relieving at least one of cough or upper respiratory symptoms, comprising orally administering an effective amount of the composition of claim 1 to a subject in need thereof.
